(19) United States
(12) Patent Application Publication
(10) Pub. No.: US 2017/0181717 A1
(43) Pub. Date: Jun. 29, 2017

(54) DUAL-PLANE X-RAY IMAGING APPARATUS

(71) Applicant: BEIJING EAST WHALE IMAGE TECHNOLOGY CO., LTD., Beijing (CN)

(72) Inventors: Chunlei TIAN, Yizhuang, Beijing (CN); Jun ZHANG, Yizhuang, Beijing (CN); Liu CAO, Yizhuang, Beijing (CN)

(21) Appl. No.: 15/304,928

(22) PCT Filed: Mar. 4, 2015
(86) PCT No.: PCT/CN2015/000131

§ 371 Date: Mar. 16, 2017

(30) Foreign Application Priority Data
Apr. 22, 2014 (CN) 201410161383.0

(51) Int. Cl. A61B 6/00

(52) U.S. Cl. CPC A61B 6/4441; A61B 6/4014; A61B 6/4405; A61B 6/4452; A61B 6/4464

(57) ABSTRACT

A biplanar X-ray imaging device includes a second C-shaped arm and a first C-shaped arm which can be slidably disposed on the second C-shaped arm; a first X-ray generation device and a first image receiving device are disposed on the first C-shaped arm, thus constituting the first imaging device; a second X-ray generation device and a second image receiving device are disposed on the second C-shaped arm, thus constituting the second imaging device; the first C-shaped arm and the second C-shaped arm share the same rotating axis. For the biplanar X-ray imaging device, when the first C-shaped arm slides along the second C-shaped arm, different angles are formed between the first imaging device and the second imaging device; the first C-shaped arm and the second C-shaped arm share the same rotating axis, which ensures that the first imaging device and the second imaging device share the same rotating axis.

### FIELD OF TECHNOLOGY

This invention relates to an X-ray imaging device, especially a biplanar X-ray imaging device.

### BACKGROUND

The biplanar imaging device has many advantages such as determining the position of patients more accurately, saving the time of operation for doctors, reducing the exposure dose and maintaining the sterile environment, so it is widely used in operations.

A biplanar imaging device can be realized through the device in U.S. Pat. No. 4,884,293 which describes a biplanar imaging system where one imaging system is fixed on the ground of the operating room and the other imaging system is hanged on the guide rail of the ceiling. The disadvantage of the device is that the two imaging systems should be matched accurately upon initial installation, which may easily lead to the result that the two imaging systems have different rotating axes.

The said biplanar imaging device can also be realized through the device in U.S. Pat. No. 3,549,885 which describes a biplanar imaging system where two imaging systems are installed in a rotatable circular ring orthogonally. The disadvantage of this device is that the angle between the two imaging systems is always 90 degrees and can't be changed.

The said biplanar imaging device can also be realized through the device in U.S. Pat. No. 5,095,501 where two imaging systems are installed in a rotatable ¾ circular ring, orthogonally to form a rotatable G-shaped arm. The disadvantage of this device is that the angle between the two imaging systems is arrays 90 degrees and can't be changed.

### SUMMARY OF THE INVENTION

This invention is designed to provide a biplanar X-ray imaging device which can form biplanar and multi-angle images of objects through the interactions between two imaging systems and their rotations and movements as a whole.

This invention adopts the following technical solutions to solve the technical problems: a biplanar X-ray imaging device, comprising the first C-shaped arm, the second C-shaped arm, a sliding and rotating seat, the first X-ray generation device, the first image receiving device, the second X-ray generation device and the second image receiving device;

The first C-shaped arm is slidably disposed on the second C-shaped arm through supporting devices; the second C-shaped arm is slidably disposed on the sliding and rotating seat;

The first X-ray generation device is fixed on one end in the peripheral direction of the first C-shaped arm;

The first image receiving device is disposed on the other end in the peripheral direction of the first C-shaped arm or slidably disposed on the inner side of the second C-shaped arm;

The second X-ray generation device and the second image receiving device are respectively fixed on the two ends in the peripheral direction of the second C-shaped arm. The intersecting line between the X-ray emission plane of the first X-ray generation device and the X-ray emission plane of the second X-ray generation device is the rotating axis;

The rotating axis is on the connecting line of the first X-ray generation device and the first image receiving device. In addition, the second X-ray generation device and the second image receiving device rotate along the rotating axis under the drive of the second C-shaped arm.

Alternatively, both the first C-shaped arm and the second C-shaped arm are hollow rectangles, and there are four supporting devices between the first C-shaped arm and the second C-shaped arm.

Alternatively; the section of the second C-shaped arm is “␣”; the section of the first C-shaped arm is “ [symbol] ”; there are four supporting devices between the first C-shaped arm and the second C-shaped arm.

Alternatively; the section of the second C-shaped arm is “␣”; the section of the first C-shaped arm is “ [symbol] ”; there are three supporting devices between the side of the second C-shaped arm and the inner surface of first C-shaped arm.

Alternatively, the section of the second C-shaped arm is “ [symbol] ”; the section of the first C-shaped arm is “ [symbol] ”; there are five supporting devices between the inner surfaces of the second C-shaped arm and the first C-shaped arm.

Alternatively, the first C-shaped arm consists of two C-shaped beams or is a C-shaped rod; a chute which matches the C-shaped beam or C-shaped rod is disposed on the second C-shaped arm.

Alternatively, the biplanar X-ray imaging device also comprises: a driving device disposed on the second C-shaped arm to drive the first C-shaped arm to slide along the second C-shaped arm;

Alternatively, there are two driving devices respectively disposed on the second C-shaped arm to drive the first C-shaped arm and the first image receiving device respectively to slide along the second C-shaped arm.

Alternatively, the biplanar X-ray imaging device also comprises lifting and rotating components, an operating arm and a mobile trolley; the sliding and rotating seat is disposed on the operating arm and can slide and rotate relative to the operating arm; the operating arm is disposed on the mobile trolley through the lifting and rotating components

Alternatively, the biplanar X-ray imaging device also comprises lifting and rotating components, an operating arm and the first base; the sliding and rotating seat is disposed on the operating arm and can slide and rotate relative to the operating arm; the operating arm is disposed on the first base through the lifting and rotating components.

Alternatively, the biplanar X-ray imaging device also comprises lifting and rotating components, an operating arm and the second base; the sliding and rotating seat is disposed on the operating arm and can slide and rotate relative to the operating arm; the operating arm is disposed on the second base through the lifting and rotating components; the second base is slidably disposed on the ceiling.

This invention has the following advantageous effects: the biplanar X-ray imaging device of this invention comprises the second C-shaped arm and the first C-shaped arm which can be slidably disposed within the second C-shaped arm; the first X-ray generation device and the first image receiving device are installed on the two ends of the first C-shaped arm, or the first X-ray generation device is installed on one end of the first C-shaped arm and the first image receiving device is disposed on the inner side of the second C-shaped arm; the first X-ray generation device and the first image receiving device constitute the first imaging device; the second X-ray generation device and the second image receiving device are installed on both ends of the second C-shaped arm; the second X-ray generation device and the second image receiving device constitute the second imaging device; when the first C-shaped arm slides along the second C-shaped arm, different angles are formed between the first imaging device and the second imaging device, so that images of the detected object can be formed from different angles; the first C-shaped arm and the second C-shaped arm share the same rotating axis, which ensures that the first imaging device and the second imaging device share the same rotating axis so that biplanar and motile-angle images of the object can be formed.

### BRIEF DESCRIPTION OF THE DRAWINGS

FIG. 1 shows the structure of the first embodiment of the biplanar X-ray of this invention;

FIG. 2 shows the structure of the second embodiment of the biplanar X-ray of this invention;

FIG. 3 shows the structure of the third embodiment of the biplanar X-ray of this invention;

FIG. 4 shows the structures of the first C-shaped arm and the second C-shaped arm of the biplanar X-ray of this invention;

FIG. 5 shows the another structures of the first C-shaped arm and the second C-shaped arm of the biplanar X-ray of this invention;

FIG. 6 shows the structure of the first embodiment of the first C-shaped arm and the section of the second C-shaped arm of the biplanar X-ray of this invention;

FIG. 7 shows the structure of the second embodiment of the first C-shaped arm and the section of the second C-shaped arm of the biplanar X-ray of this invention;

FIG. 8 shows the structure of the third embodiment of the first C-shaped arm and the section of the second C-shaped arm of the biplanar X-ray of this invention;

FIG. 9 shows the structure of the fourth embodiment of the first C-shaped arm and the section of the second C-shaped arm of the biplanar X-ray of this invention,

Labels in figures: 1—the first C-shaped arm; 2—the second C-shaped arm; 3—the sliding and rotating seat; 4—the first X-ray generation device; 5—the first image receiving device; 6—the second X-ray generation device; 7—the second image receiving device; 8—supporting device; 9—driving device; 10—lifting and rotating components; 11—operating arm; 12—mobile trolley; 13—the first base; 14—the second base.

### DETAILED DESCRIPTION OF PREFERRED EMBODIMENTS

The following part further clarifies the technical solutions of this invention in combination with embodiments and attached figures.

### Embodiment 1

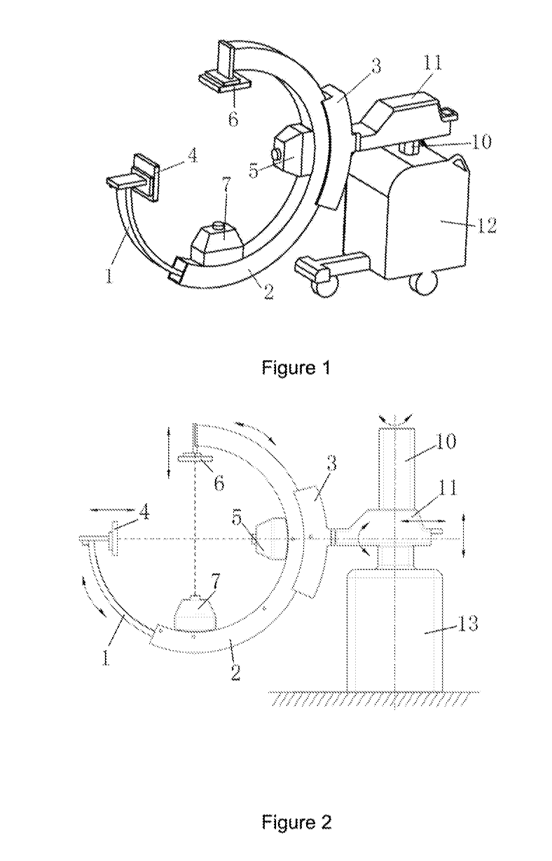

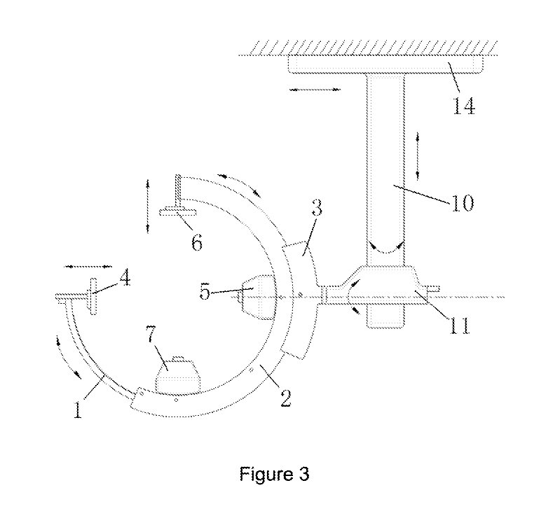

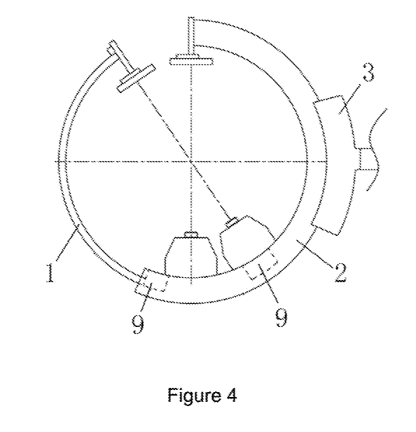

See FIGS. 1-9. This embodiment provides a biplanar X-ray imaging device, comprising the first C-shaped arm 1, the second C-shaped arm 2, the sliding and rotating seat 3, the first X-ray generation device 4, the first image receiving device 5, the second X-ray generation device 6 and the second image receiving device 7;

The first C-shaped arm 1 is slidably disposed on the second C-shaped arm 2 through the supporting device 8; the second C-shaped arm 2 is slidably disposed on the sliding and rotating seat 3;

The first X-ray generation device 4 is fixed on one end in the peripheral direction of the first C-shaped arm 1;

The first image receiving device 5 is disposed on the other end in the peripheral direction of the first C-shaped arm 1 or slidably disposed on the inner side of the second C-shaped arm 2;

The second X-ray generation device 6 and the second image receiving device 7 are respectively fixed on the two ends in the peripheral direction of the second C-shaped arm a. The intersecting line between the X-ray emission plane of the first X-ray generation device 4 and the X-ray emission plane of the second X-ray generation device 6 is the rotating axis;

The rotating axis is on the connecting line of the first X-ray generation device 4 and the first image receiving device 5. In addition, the second X-ray generation device 6 and the second image receiving device 7 rotate along the rotating axis under the drive of the second C-shaped arm 2.

The biplanar X-ray imaging device of this invention comprises: the second C-shaped arm 2 and the first C-shaped arm 1 which can be slidably disposed within the second C-shaped arm 2; the first X-ray generation device 4 and the first image receiving device 5 are installed on the two ends of the first C-shaped arm 1, or the first X-ray generation device 4 is installed on one end of the first C-shaped arm 1 and the first image receiving device 5 is disposed on the inner side of the second C-shaped arm 2; the first X-ray generation device 4 and the first image receiving device 5 constitute the first imaging device; the second X-ray generation device 6 and the second image receiving device 7 are installed on both ends of the second C-shaped arm 2; the second X-ray generation device 6 and the second image receiving device 7 constitute the second imaging device; when the first C-shaped arm 1 slides along the second C-shaped arm 2, different angles are formed between the first imaging device and the second imaging device, so that images of the detected object can be formed from different angles; the first C-shaped arm 1 and the second C-shaped arm 2 share the same rotating axis, which ensures that the first imaging device and the second imaging device share the same rotating axis so that biplanar and motile-angle images of the object can be formed.

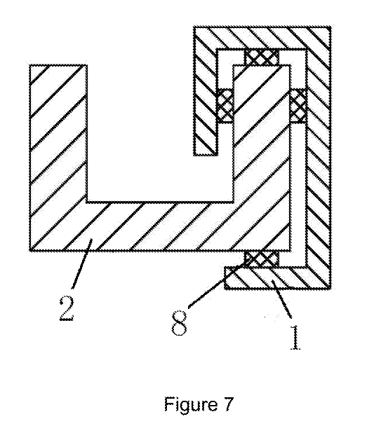

See FIG. 6. In this embodiment, alternatively, both the sections of the first C-shaped arm 1 and the second C-shaped arm 2 are rectangles, and there are four supporting devices 8 between the first C-shaped arm 1 and the second C-shaped arm 2. The four supporting devices 8 are respectively disposed on the outer surface of the first C-shaped arm 1, and the four supporting devices 8 are respectively disposed on the four sides of the rectangle of the first C-shaped arm 1, so that the second C-shaped arm 2 can support the first C-shaped arm 1 through the four supporting devices 8; besides, with the selection of materials of the supporting devices 8, such as ceramic, the supporting devices 8 can produce the technical effect of friction reduction.

See FIG. 7. In this embodiment, alternatively, the section of the second C-shaped arm is “␣”; the section of the first C-shaped arm is “ [symbol] ”; there are four supporting devices between the first C-shaped arm and the second C-shaped arm; the four supporting devices are disposed in the position between the part of the second C-shaped arm which is within the first C-shaped arm and the first C-shaped arm, i.e. the four sides (upside, downside, left side and right side) of the part of the second C-shaped arm which is within the first C-shaped arm; the second C-shaped arm support the first C-shaped arm through the four supporting devices; besides, with the selection of materials of the supporting devices, such as ceramic, the supporting devices can produce the technical effect of friction reduction.

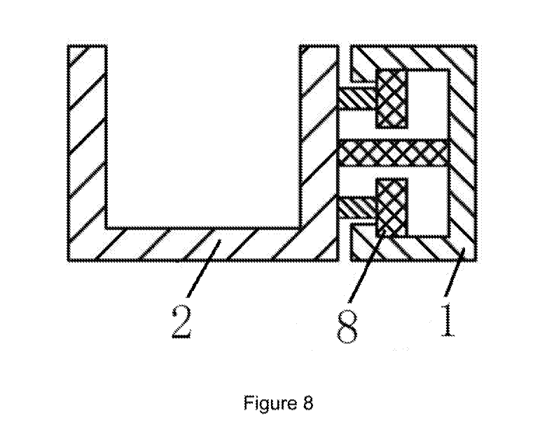

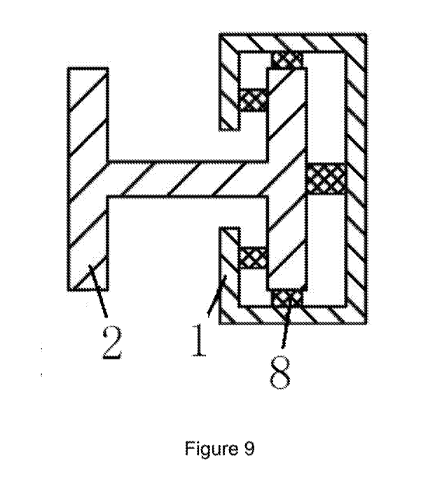

See FIG. 8. In this embodiment, alternatively, the section of the second C-shaped arm is “␣”; the section of the first C-shaped arm is “ [symbol] ”, i.e. a rectangle with an opening on its side; there are two protruding parts on the side with an opening; there are three supporting devices between the side of the second C-shaped arm and the inner surface of the first C-shaped arm; two of the three supporting devices are respectively disposed on the side of the second C-shaped arm and stuck in the protruding parts; the third supporting device is disposed between the two supporting devices, so that the second C-shaped arm applies a pulling force to the first C-shaped arm through the two supporting devices and the second C-shaped arm applies pressure on the first C-shaped arm through the third supporting device.

See FIG. 9. In this embodiment, alternatively, the section of the second C-shaped arm is “ [symbol] ”; the section of the first C-shaped arm is “ [symbol] ”; there are five supporting devices between the inner surfaces of the second C-shaped arm and the first C-shaped arm; the five supporting devices are respectively disposed on the upside, downside, upper left side, lower left side and right side of the part of the second C-shaped arm which is within the first C-shaped arm; the second C-shaped arm support the first C-shaped arm through the five supporting devices; besides, with the selection of materials of the supporting devices, such as ceramic, the supporting devices can produce the technical effect of friction reduction.

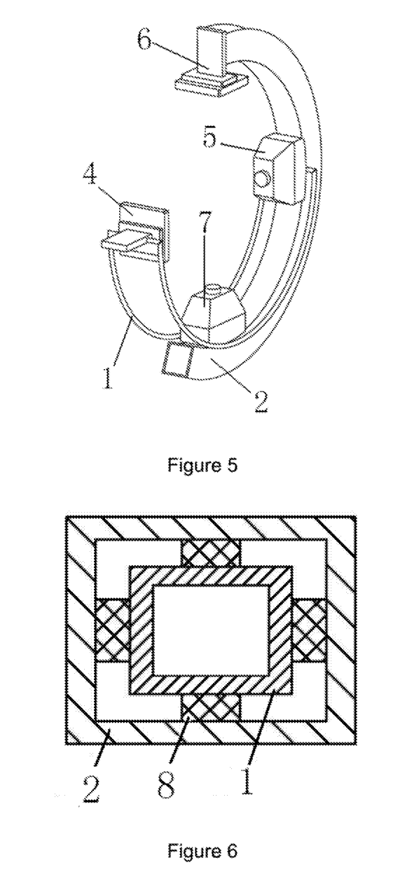

See FIG. 5 in this embodiment, alternatively, the first C-shaped arm 1 consists of two C-shaped beams or is a C-shaped rod; the second C-shaped arm 2; a chute which matches the C-shaped beam or C-shaped rod is disposed on the second C-shaped arm 2 to reduce the mass of the first C-shaped arm and enhance the accuracy of control over the first C-shaped arm, thus enhancing the precision of the biplanar X-ray imaging device.

In this embodiment, alternatively, the biplanar X-ray imaging device also comprises the driving device 9; the driving device 9 is disposed on the second C-shaped arm 2 to drive the first C-shaped arm 1 to slide along the second C-shaped arm the first C-shaped arm 1 is driven by the driving device 9;

Alternatively, there are two driving devices 9 respectively disposed on the second C-shaped arm 2 to drive the first C-shaped arm 1 and the first image receiving device 5 respectively to slide along the second C-shaped arm 2. Preferably, the driving device 9 is a motor installed with gears on its rotor shaft; gear teeth which match the gears are disposed on the inner side of the first C-shaped arm 1, so that the first C-shaped arm is driven by the motor.

See FIG. 1. In this embodiment, alternatively, the biplanar X-ray imaging device also comprises lifting and rotating components 10, an operating arm 11 and a mobile trolley 12; the sliding and rotating seat 3 is disposed on the operating arm 11 and can slide and rotate relative to the operating arm 11; the operating arm 11 is disposed on the mobile trolley 12 through the lifting and rotating components 10 to realize the multi-degree-of-freedom control over the first image receiving device 5 and the second image receiving device 6.

See FIG. 2. In this embodiment, alternatively, the biplanar X-ray imaging device also comprises lifting and rotating components 10, an operating arm 11 and the first base 13; the sliding and rotating seat 3 is disposed on the operating arm 11 and can slide and rotate relative to the operating arm 11; the operating arm 11 is disposed on the first base 13 through the lifting and rotating components 10 to realize the multi-degree-of-freedom control over the first image receiving device 5 and the second image receiving device 6.

In this embodiment, alternatively, the biplanar X-ray imaging device also comprises lifting and rotating components 10, an operating arm 11 and the second base 14; the sliding and rotating seat 3 is disposed on the operating arm 11 and can slide and rotate relative to the operating arm 11; the operating arm 11 is disposed on the second base 14 through the lifting and rotating components 10; the second base 14 is slidably disposed on the ceiling to realize the multi-degree-of-freedom control over the first image receiving device 5 and the second image receiving device 6.

The order of the above embodiments is arranged for the convenience of description and doesn't represent the priority of these embodiments.

Finally, it should be noted that: the above embodiments are used to illustrate the technical solutions of this invention only instead of limiting the scope of this invention. Although a detailed explanation has been made to this invention with reference to above-mentioned embodiments, common technicians in the field should understand that: they can modify the technical solutions recorded in the above-mentioned embodiments or replace some technical features therein with equivalents; these modifications or replacements shall not cause the nature of technical solutions to deviate from the spirit and scope of technical solutions of each embodiment of this invention.

1. A biplanar X-ray imaging device, characterized in comprising a first C-shaped arm, a second C-shaped arm, a sliding and rotating seat, a first X-ray generation device, a first image receiving device, a second X-ray generation device and a second image receiving device;
the first C-shaped arm is slidably disposed on the second C-shaped arm through supporting devices; the second C-shaped arm is slidably disposed on the sliding and rotating seat;
the first X-ray generation device is fixed on one end in the peripheral direction of the first C-shaped arm;
the first image receiving device is disposed on the other end in the peripheral direction of the first C-shaped arm or slidably disposed on the inner side of the second C-shaped arm;
the second X-ray generation device and the second image receiving device are respectively fixed on the two ends in the peripheral direction of the second C-shaped arm; the intersecting line between the X-ray emission plane of the first X-ray generation device and the X-ray emission plane of the second X-ray generation device is the rotating axis;
the rotating axis is on the connecting line of the first X-ray generation device and the first image receiving device; addition, the second X-ray generation device and the second image receiving device rotate along the rotating axis under the drive of the second C-shaped arm.

2. A biplanar X-ray imaging device according to claim 1, characterized in that both the first C-shaped arm and the second C-shaped arm are hollow rectangles, and there are four supporting devices between the first C-shaped arm and the second C-shaped arm.

3. A biplanar X-ray imaging device according to claim 1, characterized in that the section of the second C-shaped arm is “␣”; the section of the first C-shaped arm is “ [symbol] ”; there are four supporting devices between the first C-shaped arm and the second C-shaped arm.

4. A biplanar X-ray imaging device according to claim 1, characterized in that the section of the second C-shaped arm is “␣”; the section of the first C-shaped arm is “ [symbol] ”; there are three supporting devices between the side of the second C-shaped arm and the inner surface of first C-shaped arm.

5. A biplanar X-ray imaging device according to claim 1, characterized in that the section of the second C-shaped arm is “ [symbol] ”; the section of the first C-shaped arm is “ [symbol] ”; there are five supporting devices between the inner surfaces of the second C-shaped arm and the first C-shaped arm.

6. A biplanar X-ray imaging device according to claim 1, characterized in that the first C-shaped arm consists of two C-shaped beams or is a C-shaped rod; a chute which matches the C-shaped beam or C-shaped rod is disposed on the second C-shaped arm.

7. A biplanar X-ray imaging device according to claim 1, characterized in that the biplanar X-ray imaging device also comprises a driving device; the driving device is disposed on the second C-shaped arm to drive the first C-shaped arm to slide along the second C-shaped arm;
alternatively there are two driving devices respectively disposed on the second C-shaped arm to drive the first C-shaped arm and the first image receiving device respectively to slide along the second C-shaped arm.

8. A biplanar X-ray imaging device according to claim 7, characterized in that the biplanar X-ray imaging device also comprises lifting and rotating components, an operating arm and a mobile trolley; the sliding and rotating seat is disposed on the operating arm and can slide and rotate relative to the operating arm; the operating arm is disposed on the mobile trolley through the lifting and rotating components.

9. A biplanar X-ray imaging device according to claim 7, characterized in that the biplanar X-ray imaging device also comprises lifting and rotating components, an operating arm and the first base; the sliding and rotating seat is disposed on the operating arm and can slide and rotate relative to the operating arm; the operating arm is disposed on the first base through the lifting and rotating components.

10. A biplanar X-ray imaging device according to claim 7, characterized in that the biplanar X-ray imaging device also comprises lifting and rotating components, an operating arm and the second base; the sliding and rotating seat is disposed on the operating arm and can slide and rotate relative to the operating arm; the operating arm is disposed on the second base through the lifting and rotating components; the second base is slidably disposed on the ceiling.
